SEQUENCE LISTING (3 sequences)

SEQ ID NO: 1 - RNA, 19 nt, Artificial Sequence
  siRNA specific to BRCA2 (SiBRCA2 #1)
gucagugguaugugggagu

SEQ ID NO: 2 - RNA, 19 nt, Artificial Sequence
  siRNA specific to BRCA2 (SiBRCA2 #2)
uaguaggauauuguucuuc

SEQ ID NO: 3 - DNA, 20 bp, Artificial Sequence
  DNA sequence complementary to siRNA specific to BRCA2
aaatgacctactggcacttt

(19) United States
(12) Patent Application Publication
(10) Pub. No.: US 2008/0102477 A1
(43) Pub. Date: May 1, 2008

(54) Method for screening an anticancer drug using acetylated BubR1

(75) Inventors: Hyunsook Lee, Seoul (KR); Eunhee Choi, Seoul (KR); Ji yoon Choi, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Seoul National University Industry Foundation

(21) Appl. No.: 11/585,811

(22) Filed: Oct. 25, 2006

(51) Int. Cl. G01N 33/574; C07K 16/30

(52) U.S. Cl. 435/7.23; 435/320.1; 530/388.8

(57) ABSTRACT

A method of screening for an anticancer drug comprising treating a cancer cell with candidate compounds and assessing the change in acetylated BubR1 level of the cancer cell is useful for efficiently screening an anticancer drug. An antibody which specifically binds to the 250^{th} amino acid residue, Lys, of BubR1 and an expression plasmid for an animal cell comprising a DNA encoding a fusion protein of BubR1 and a marker protein are also disclosed.

### FIELD OF THE INVENTION

The present invention relates to a method of screening for an effective anticancer drug comprising treating a cancer cell with candidate compounds and assessing the level of acetylated BubR1 in the cancer cell.

### BACKGROUND OF THE INVENTION

It has recently been reported that the generation of aneuploid during mitosis plays an important role in the initial stage of cancerization and the most malignant tumor cells are aneuploid (Pihan G. A. et al., Cancer Biology, 9: 289-302 (1999)). This suggests that the generation of aneuploid is a general phenomenon when a normal cell undergoes transformation to a cancer cell.

Through many in vitro and in vivo experimental results obtained for human cells or animal cell, it is clearly established that aneuploid is directly related to neoplastic transformation and immortalization of cells. It is well known that aneuploid can be induced by chemical carcinogens, ionizing radiation or gene mutations. It has been also reported that structural abnormality of cells and alteration of cell growth directly affect genome, causing chromosomal instability (CIN) (Hannahan, D. and R. A. Weinberg, Cell, 100: 57-70 (2000); Ruhong Li. et al., PNAS, 97: 3236-3241 (2000)).

It is well established that DNA damage induces G2 arrest in interphase cells. However, when dividing cells encounter DNA breaks, mitotic arrest is induced via the activation of spindle checkpoint (A. Mikhailov et al., Curr Biol., 12: 1797 (2002)). If this fails, cells will acquire aneuploidy.

The present inventors have discovered that acetylated BubR1 inhibits the generation of aneuploid by playing an important role in activating spindle checkpoint, and if BubR1 fails to be acetylated in cells, the BubR1 level decreases immediately due to its ubiquitination-dependent proteolysis and the cells get aneuploid.

### SUMMARY OF THE INVENTION

Accordingly, it is an object of the present invention to provide a method for screening anticancer drugs using acetylated BubR1.

In accordance with one aspect of the present invention, there is provided a method of screening for an effective anticancer drug comprising treating a cancer cell with candidate compounds and assessing the level of acetylated BubR1 in the cancer cell.

### BRIEF DESCRIPTION OF THE DRAWINGS

The above and other objects and features of the present invention will become apparent from the following description of the invention, when taken in conjunction with the accompanying drawings which respectively show:

FIG. 1: the result of Western blot analysis (WB) showing that BubR1 is acetylated after DNA damage in a cell;

FIG. 2: the result of WB showing that BRCA2 and BubR1 formed a complex after DNA damage in a cell;

FIG. 3: the result of WB showing increased BubR1 acetylation by PCAF in the presence of BRCA2;

FIG. 4: the result of WB showing BRCA2-dependant association between BubR1 and PCAF;

FIG. 5: the result of WB showing that the level of acetylated BubR1 in a cell decreases following knockdown of BRCA2 expression;

FIG. 6: the result of WB showing the inhibition of BubR1 ubiquitination by BRCA2;

FIG. 7: the result of Mass Spectrometry identifying the acetylation site of BubR1;

FIG. 8: the result of the WB showing intracellular BubR1 level in case of K250R (the 250^{th} amino acid of BubR1, lysine, is replaced by arginine) and K250Q (250^{th} amino acid of BubR1, lysine, is replaced by glutamine);

FIG. 9: the result of WB comparing ubiquitination of K250R, wild-type BubR1 and K250Q; and

FIG. 10: the result of DAPI-staining for counting the number of apoptotic cells.

FIG. 11: the structure of plasmid pDsRed express-C1 BubR1.

### DETAILED DESCRIPTION OF THE INVENTION

When DNA is severely damaged, the breast cancer associated protein, BRCA2, forms a complex with the checkpoint protein, BubR1, at the kinetochore in prometaphase-arrested cell. Specifically, the C-terminal of BRCA2 binds to the N-terminal of BubR1. Then, BRCA2, in the form of a complex with PCAF (histone acetyltransferase), acetylates BubR1 at the 250th amino acid residue, lysine (K250), and inhibits proteolysis of BubR1. The acetylated BubR1 takes a crucial role in the process of inducing mitotic arrest via the activation of spindle checkpoint. In BRCA2-deficient cells, BubR1 is lost from the kinetochore and this is correlated with chromosome mis-segregation. Thus, BRCA2 coordinates DNA damage with the spindle checkpoint owing to its ability to form a complex with PCAF, which acetylates BubR1. Mutation of BRCA2 accumulates defects in DNA repair and mitotic checkpoint control, resulting in aneuploidy with aberrant chromosome translocation. Meanwhile, HDAC (histone deacetylase) removes the acetyl group from acetylated BubR1 in the cell and free BubR1 is confronted with ubiquitination, resulting in the proteolysis of BubR1.

The above-mentioned mechanism of interaction between BRCA2 and BubR1 are conserved in vertebrates.

The present inventors have discovered the above-mentioned molecular mechanism for the first time and the inventive screening method is based on this mechanism.

The screening method of the present invention may comprise:

a) introducing an expression plasmid comprising a DNA sequence encoding a fusion protein of BubR1 and a marker protein into a cancer cell expressing BRCA2 and PCAF;

b) treating the cancer cell with candidate compounds;

c) assessing the level of acetylated BubR1 in the cancer cell by fluorescence analysis for the marker protein; and

d) selecting the compound which enhances the level of acetylated BubR1 as an anticancer drug.

Preferable cancer cell lines for use in the inventive method include, but not limited to, HeLa, MCF7, T98G, U20S and Saos2.

The expression plasmid may be prepared by inserting a DNA encoding a fusion protein of BubR1 and a marker protein into a conventional eukaryotic expression plasmid comprising a promoter working in eukaryotic cells and an antibiotics resistance gene.

The marker protein may be any fluorescence protein, examples thereof including Green Fluorescence Protein (GFP) and Red Fluorescence Protein (RFP). The fluorescence analysis may be conducted by a conventional method such as fluorescence microscopy.

Transformation can be conducted by using a commercially available materials for transfection, such as Effectene® (Qiagen), Lipofectamin® (Invitrogen) and Fugene®6 (Roche), or by electroporation.

The inventive screening method may also be conducted by assessing the level of acetylated BubR1 by ELISA using an antibody specific for BubR1 acetylated at K250. Specifically, this method comprises: a) treating a cancer cell with candidate compounds, b) assessing the level of acetylated BubR1 in the cancer cell by ELISA using an antibody specific for acetylated BubR1, and c) selecting the compound which enhances the level of acetylated BubR1 as an anticancer drug.

The antibody specific for acetylated BubR1 may be prepared by conjugating acetylated BubR1 with keyhole limpet hemocyanin (KLH) or bovine serum albumin (BSA), injecting the conjugate into an animal and then taking the antibodies from the blood sample of the animal.

ELISA in the present invention may be conducted in accordance with a conventional procedure, for example, by a method comprising:

a) seeding a cancer cell on each well of a microtiter plate;

b) treating the cancer cell with a candidate compound;

c) treating the cancer cell obtained in b) with a first antibody specific for acetylated BubR1;

d) treating the cancer cell obtained in c) with a horseradish peroxidase (HRP)-conjugated second antibody, and then with a substrate of HRP; and

e) assessing the level of acetylated BubR1 by measuring the intensity of the resulting HRP-substrate reaction.

The inventive screening method is useful as a high-throughput screening method for screening a number of candidate compounds in a short time.

The following Examples are intended to further illustrate the present invention without limiting its scope.

### REFERENCE EXAMPLE 1

### Preparation of Total Cell Lysate (TCL)

Cells were lysed in NETN buffer (150 mM NaCl, 1 mM EDTA, 20 mM Tris (pH 8.0), 0.5% NP-40) supplied with protease inhibitor cocktails (Roche).

### EXAMPLE 1

### Acetylation of BubR1 by BRCA2 and PCAF

(1) Acetylation of BubR1 Following DNA Damage

MCF7 cells were exposed to γ-irradiation (IR) (15-Gy) and the cells taken before and 6 hours after treating IR treatment were lysed by the method of Reference Example 1. 2 mg each of the resulting total cell lysates (TCL) was subjected to immunoprecipitation (IP) with anti-actyl-K antibodies (αAc-K) (Cell signalling). 50 μg each of TCL was subjected to SDS-PAGE and subsequent Western blot assay (WB) with anti-BubR1 antibodies (αBubR1) (BD Bioscience). The extent of DNA damage in the cells was assessed by immunoblotting with anti-phosphorylated p53 antibodies.

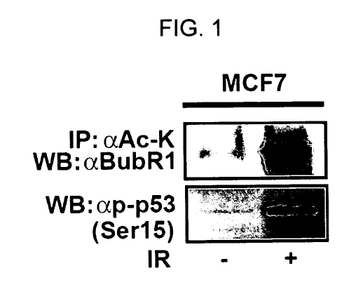

As shown in FIG. 1, the level of acetylated BubR1 increased after DNA damage induced by IR.

(2) Formation of a Complex Between BRCA2 and BubR1 Upon DNA Damage

293T cells were transfected with expression plasmids constructed by inserting DNA sequences encoding BubR1 and BRCA2, respectively into pcDNA 3.1 (Invitrogen), respectively, and then treated with 2.4 μM adriamycin (Dox) for inducing DNA damage, or left untreated. The cells were treated with 100 ng/ml of nocodazole (Noc) for the cell cycle synchronization in prometaphase, or left untreated. After 48, TCL (50 μg) were subjected to IP using anti-BRCA2 antibodies (αBRCA2) (Upstate Biotechnology), followed by WB using αBubR1. TCL was also analyzed by WB using αBubR1 and anti-Lamin A/C antibodies (αLamin A/C) (Frank Mckeon).

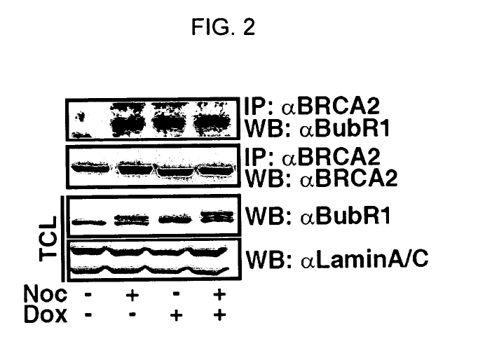

The result in FIG. 2 indicates BRCA2 and BubR1 complexes were formed in prometaphase, and following DNA damage.

(3) BubR1 Acetylation by PCAF in the Presence of BRCA2

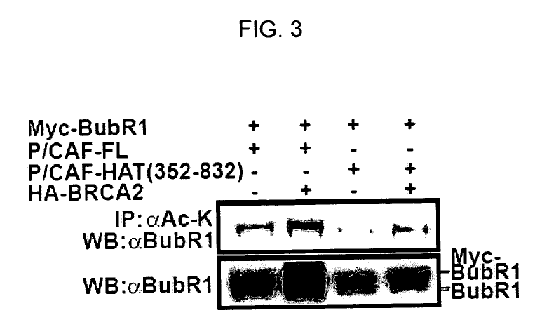

293T cells were transfected with the expression plasmids constructed by inserting DNA sequences encoding myc-BubR1(mycubiquitin-BubR1), hemaglutinin (HA)-BRCA2, PCAF-FL (full length) and PCAF352-832-FLAG3X (an N-terminal fusion of three repeats of the FLAG epitope to PCAF-HAT domain consisting of 352^{nd} to 832^{nd} amino acid of PCAF) into pcDNA 3.1 (Invitrogen), respectively. TCL (50 μg) was subjected to IP with αAc-K, followed by WB with αBubR1. In FIG. 3, Bottom panel shows the levels of endogenous BubR1 and ectopically expressed myc-BubR1.

As shown in FIG. 3, when BRCA2 is co-expressed with PCAF or PCAF HAT domain (PCAF-HAT 352-832), acetylation of BubR1 is increased.

(4) BRCA2-dependant Association Between BubR1 and PCAF

293T cells were transfected with expression plasmids constructed by inserting DNA sequences encoding myc-BubR1, HA-BRCA2 or PCAF-FL into pcDNA 3.1 (Invitrogen), respectively, and treated with 2.4 μM adriamycin (Dox) for inducing DNA damage, or left untreated. After 48 hours, TCL were subjected to IP using anti-PCAF antibodies (APCAF) (Santa Cruz Biotechnology), followed by WB using αBubR1. The control WB for the levels of PCAF in each of the sample was conducted as well.

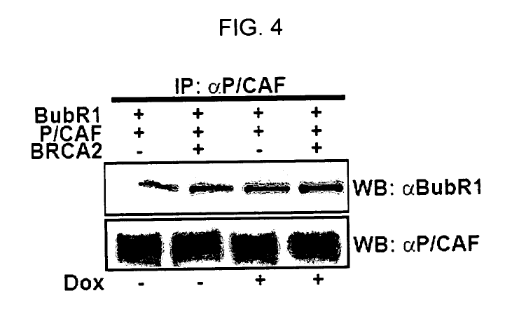

The result in FIG. 4 indicates that BubR1 associated with PCAF and the level of BubR1 was increased in the presence of overexpressed BRCA2 and upon DNA damage. This result implies that upon DNA damage, BRCA2 recruits PCAF having HAT activity and acetylates BubR1.

(5) The Effect of Knockdown of BRCA2 Expression

Two different synthetic siRNAs specific to BRCA2, SiBRCA2 #1 (GUCAGUGGUAUGUGGGAGU; SEQ ID NO: 1) and SiBRCA2 #2 (UAGUAGGAUAUUGUUCUUC; SEQ ID NO: 2) for BRCA2 were introduced into MCF7 cells by employing oligofectamine (Invitrogen), and TCL were subjected to IP using αAc-K, followed by WB using αBubR1. TCL was also analyzed by WB using αBRCA2, αBubR1 and anti-Actin antibodies (αActin).

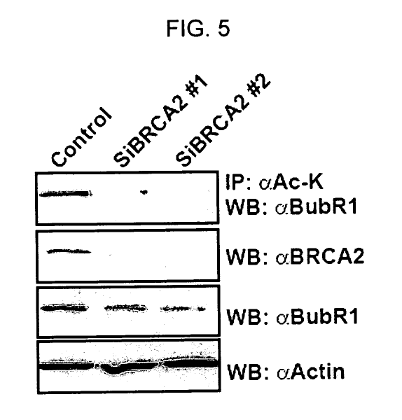

As shown in FIG. 5, knockdown of BRCA2 expression by the use of siRNA significantly reduced the level of acetylation of BubR1, as well as BubR1 protein. This result implies that BRCA2 regulates acetylation of BubR1 and stability of BubR1 is due to its acetylation.

### EXAMPLE 2

### Inhibition of BubR1 Ubiquitination by BRCA2

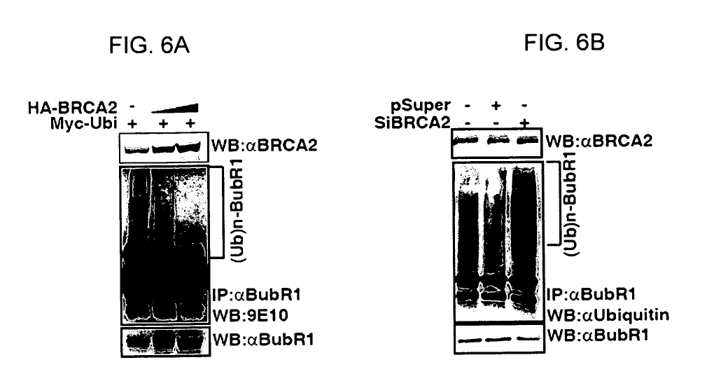

A plasmids constructed by inserting a DNA encoding ubiquitin fused to the myc epitope (myc-ubiquitin) and BubR1, respectively into pcDNA3.1 was transfected into 293T cells alone, or in combination with an HA-BRCA2 expression plasmid. TCL was subjected to IP using αBubR1, and analyzed by WB with anti-myc antibodies, 9E10, obtained from cell line 9E10 (ATCC CRL-1729) to determine the extent of polyubiquitination of BubR1 ((Ub)n-BubR1) (FIG. 6A). It was confirmed that similar amounts of BubR1 immunoprecipitates were subjected for WB analysis with αBubR1(FIG. 6A, bottom panel).

Further, pSuper-BRCA2 expressing siRNA (SiBRCA2) specific to BRCA, was constructed by inserting duplex oligos (AAATGACCTACTGGCACTTT; SEQ ID NO: 3) into pSuper vector (OligoENgine, WA) according to the manufacturer's recommendation and introduced into the MCF7 cells by employing Effectene® (Qiagen). As a control, pSuper vector was also introduced into the MCF7 cells. TCL was subjected to IP using αBubR1, and analyzed by WB with anti-ubiquitin antibodies (aubiquitin) to determine the extent of polyubiquitination of BubR1 ((Ub)n-BubR1). Same blot was re-probed with anti-BubR1 antibody to verify the immunoprecipitated level (FIG. 6B, bottom panel).

As shown in FIGS. 6A and 6B, polyubiquitination of BubR1, which targets the protein for proteosome-dependent degradation, was markedly reduced by overexpression of BRCA2, while knock-down of BRCA2 expression by siRNA (pSuper-BRCA2) results in an increase in the level of ubiquitination of endogenous BubR1 (FIG. 6B).

### EXAMPLE 3

### In Vitro Acetylation Assay and Mass Spectrometry

BubR1 (amino acids 1-514) and PCAF (amino acids 352-832) were subcloned into pGEX 5X-1 (Amersham) and purified as GST-fusion proteins from E. coli, respectively. The resulting recombinant BubR1 protein was incubated with or without the resulting recombinant PCAF protein for 1 hour at 30° C. in HAT buffer: 250 mM Tris-HCl, pH 8.0; 50% glycerol; 0.5 mM EDTA; 5 mM dithiothreitol and 1 mM unlabelled acetyl-coenzyme A. Proteins were separated by SDS-PAGE and analyzed by Western blot using an anti-acetyl lysine antibodies (Upstate Biotechnology) to visualize acetylated BubR1 (amino acids 1-514). Matrix-assisted laser desorption/ionization mass spectrometry (MALDI-MS) was carried out using a delayed-extraction reflectron time-of-flight mass spectrometer (Model M@LDI-R; Micromass, Manchester, UK). Acetylated peptides were generated by in-gel digestion using pepsin, and MS/MS analysis was carried out by nanoflow electrospray ionization (nano-ESI) on a Q-TOF2 mass spectrometer (Micromass, Manchester, UK). Acetylated peptides were identified by matching peptide masses from MALDI-TOF MS with theoretical peptides derived from proteins in the NCBI database using MASCOT® software and Profound software.

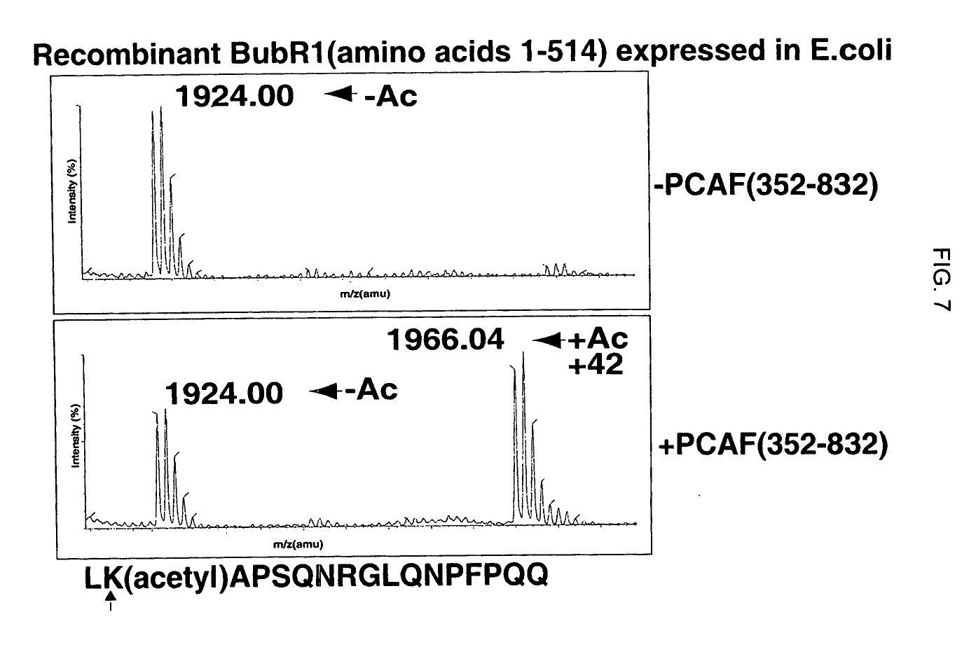

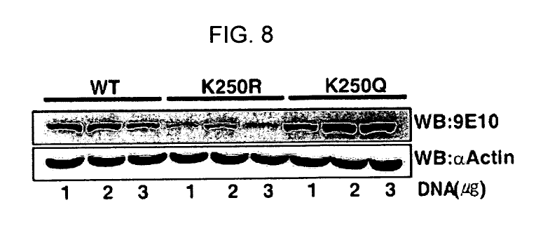

As shown in FIG. 8, the appearance of a 42 Da acetylation peak in the presence of active PCAF (PCAF 352-832) confirmed that lysine 250 (K250) is the acetylation site of BubR1 in the cell (FIG. 7). It is also confirmed that BubR1 sequences surrounding K250 among various species are conserved, specifically Destruction Box domain important to proteolysis in mitotic cyclinB is adjacent to the sequences.

### EXAMPLE 4

### Identification of an Acetylation Site of BubR1

The acetylation-defective mutant of BubR1, K250R (the 250^{th} amino acid, lysine, is replaced by arginine) and K250Q (the 250^{th} amino acid, lysine, is replaced by glutamine), was prepared by site-directed mutagenesis using pcDNA3.1 (Invitrogen)-myc-BubR1 prepared in (3) of Examples 2 as a template. K250Q was expected to mimic acetylated BubR1 because its structure is similar to acetylated BubR1. Increasing amounts of expression plasmids having wild-type BubR1-, K250R- or K250Q-encoding DNA were transfected into 293T cells and TCL was analyzed by WB using the 9E10 and αActin.

As shown in FIG. 8, the level of BubR1 protein was significantly lower in K250R-transfected cells, compared to wild type or K250Q-transfected cells. This result suggests that acetylation of BubR1 at lysine 250 is responsible for proteolysis of BubR1.

### EXAMPLE 5

### Ubiguitination of Wild-Type BubR1 K250R and K2500

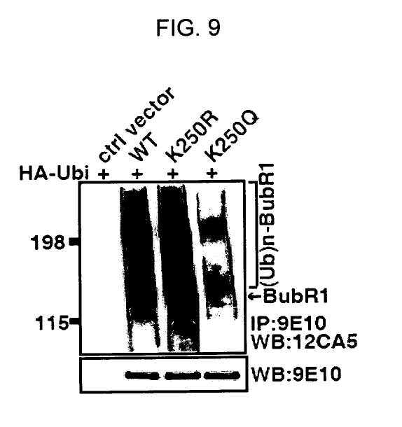

Expression plasmids having wild-type BubR1-, K250R- or K250Q-encoding DNA sequences prepared in Example 4 were transfected into 293T cells, respectively, together with a plasmids constructed by inserting DNA encoding HA-ubiquitin (hemaglutinin-ubiquitin). TCL obtained from the transfected cells was subjected to immunoprecipitation with 9E10, and analyzed by WB using a anti-hemaglutinin antibodies (12CA5) (Frank Mckeon) to detect poly-ubiquitination of BubR1 ((Ub)n-BubR1). The blot was re-probed with 9E10 to control for the amount of immunoprecipitated BubR1 in each sample. As shown in FIG. 9, ubiquitination of acetylation-defective K250R was higher compared to wild-type, whereas that of K250Q was markedly reduced.

### EXAMPLE 6

### Effect of Wild-Type BubR1, K250R and K250Q Overexpression in HeLa Cells

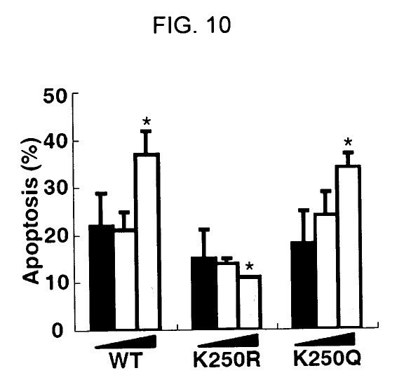

Wild-type BubR1, K250R and K250Q were overexpressed in p53-deficient HeLa cells (ATCC) and scored for apoptotic cells. Specifically, increasing amounts (3, 6, 9 μg) of each plasmid constructed by inserting a DNA sequence encoding EGFP (Enhanced Green Fluorescence Protein) fused to BubR1, K250R or K250Q was transfected into 3×10⁴ HeLa cells growing on cover slips, and the number of apoptotic cells, determined by DAPI-staining, was counted (n=200). As shown in FIG. 10, while mitotic catastrophe was not observed in K250R-overexpressing cells, excessive BubR1 or K250Q resulted in increased cell death. This result suggests that the acetylation of BubR1 in the cell takes a crucial role in the process of inducing mitotic arrest.

### EXAMPLE 7

### Preparation of Antibody of Acetylated BubR1 at K250

An antibody specific for BubR1 acetylated at K250 was prepared as the follows.

BubR1 acetylated at K250 was obtained from Thermo electron coporation (Korea), and the immunization was conducted by using Imject® Immunogen EDC kit (PIERCE) according to the manufacturer's recommendation.

2 mg of Imject® mcKLH (PIERCE) was dissolved in 200 μl of ultrapure water (autoclaved third distilled water) and 2 mg of BubR1 acetylated at K250 was dissolved in 0.5 ml of Imject® EDC conjugation buffer (PIERCE): The resulting solutions were mixed throughly. 10 mg of EDC was dissolved in 1 ml of ultrapure water, and then 50 μl of the resulting solution was added to the mixed solution obtained above, followed by incubation for 2 hours at room temperature. 60 ml of ultrapure water was added to Imject® Purification Buffer Salt (PIERCE) and purification was conducted by filtration using desalting column (0.5 ml of solution per column). The column was washed by 20 ml of Purification Buffer Salt. About 0.5 ml of the filtrated solution was put in the column and collect by 8˜10 drops. Fractions having BubR1-mcKLH was collected by absorbance analysis at 280 nm for collected fractions.

500 ml of the collected product (100 μg) and Freund's complete adjuvant were mixed (1:1) and injected into abdominal cavity of a mouse. After 14 days, the same amount of the product was obtained and mixed with Freund's Imcomlete adjuvant (1:1) and injected into abdominal cavity of the mouse again. After 14 days, about 1 ml of blood was obtained from a heart of the mouse and was kept for a day at 4° C. The blood sample was subjected to centrifugation at 12,000 rpm for 10 minutes to obtain a supernatant. Obtained serum stock was kept in −80° C. deep freezer.

### EXAMPLE 8

### Screening of Anticancer Drug I

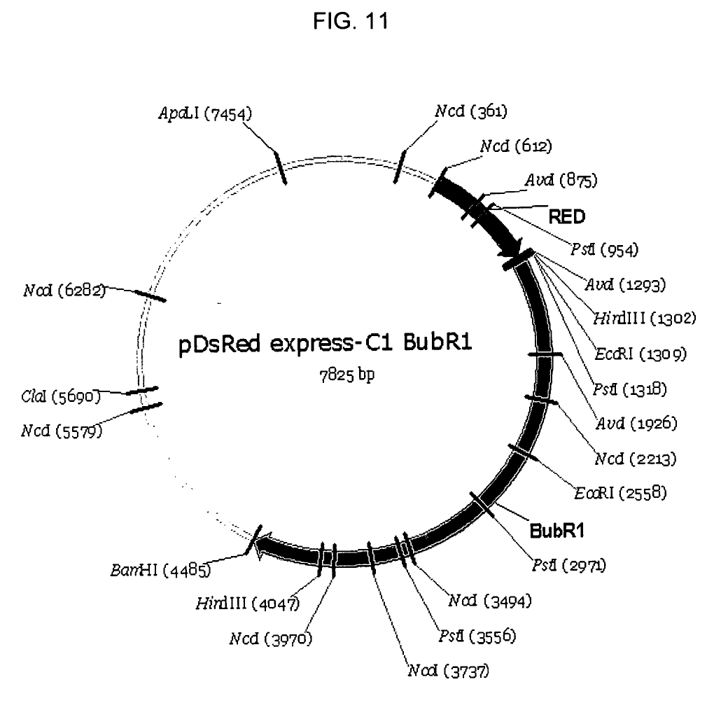

HeLa cells are transfected with plasmid, pDsRed express-C1 BubR1 having the structure shown in FIG. 11. 2×10⁵ transformed HeLa cells are cultured in a culture medium (DMEM (DULBECCO'S MODIFIED EAGLE'S MEDIUM), 10% FBS (Fetal Bovine Serum) and 1% Penicillin/Streptomycin) and put in an 5% CO₂ incubator at 36° C. After 3 hours, the cells are treated with candidate compounds, e.g., Trichostatin A (TSA) and the level of acetylated BubR1 in the cell is assessed by taking Fluorescence images of live cells at an intervals of 10 minutes fluorescence analysis. The fluorescence images (20× and 40×) are obtained by employing Delta Vision RT (Applied Precision) with dsRED (Excitation 580/20 nm; Emission 630/60 nm) filter.

### EXAMPLE 9

### Screening of Anticancer Drug II

5×10³ MCF7 cells are fixed on each well of a p6-well microtiter plate overnight at 4° C. Candidate compounds, e.g., Trichostatin A (TSA) are added to the wells at various concentrations. 3 hours after the treatment, TCL is prepared from the cells and transferred a microtiter plate wherein the wells are coated with the anti-BubR1 antibody. The plate is shaken for 30 minutes at room temperature to allow the binding of coated anti-BubR1 antibody with BubR1 in the TCL. After the plate wells, the acetylated BubR1 antibody prepared in Example 7 is added to each well and the plate is incubated at room temperature for 30 minutes. The plate wells are washed three times with PBS. Then HRP-conjugated secondary antibody (Zymed) is added to each well and the plate is incubated at room temperature for 30 minutes. The plates is washed and a substrate of HRP, Diaminobenzidine, is added to the wells. The plate is incubated at room temperature for 15 minutes with shaking. The reaction is terminated by adding a stop solution. Level of acetylated BubR1 in the cell is determined by the absorbance at 450 nm (A₄₅₀).

1. A method of screening candidate compounds for an anticancer drug comprising:
treating a cancer cell expressing breast cancer 2 (BRCA2) and p300/cAMP response element binding protein-associated factor (PCAF) with each of candidate compounds;
assessing the level of human BUB1 budding uninhibited by benzimidazole 1 homolog beta (BubR1) acetylated at the 250^{th} amino acid residue, lysine, in the cancer cell; and
selecting a compound, which enhances the level of the acetylated BubR1 in the cancer cell, as an anticancer drug.

2. The method of claim 1, which comprises:
a) introducing an expression plasmid comprising a DNA encoding a fusion protein of BubR1 and a marker protein into a cancer cell expressing BRCA2 and PCAF;
b) treating the cancer cell with each of this candidate compounds;
c) assessing the level of human BubR1 acetylated at the 250^{th} amino acid residue, lysine, in the cancer cell by fluorescence analysis for the marker protein; and
d) selecting a compound which enhances the level of acetylated BubR1 as an anticancer drug.

3. The method of claim 2, wherein the marker protein is green fluorescence protein or red fluorescence protein.

4. The method of claim 1, wherein the step of assessing the level of acetylated BubR1 acetylated at the 250^{th} amino acid residue, lysine, is carried out by ELISA using an antibody specific for acetylated BubR1.

5. (canceled)

6. An antibody which specifically binds to the 250^{th} amino acid residue, Lys, of BubR1.

7. An expression plasmid for an animal cell comprising a DNA encoding a fusion protein of BubR1 and a marker protein.

8. The expression plasmid of claim 7, wherein the marker protein is green fluorescence protein or red fluorescence protein.
